(19) United States
(12) Patent Application Publication
(10) Pub. No.: US 2023/0060820 A1
(43) Pub. Date: Mar. 2, 2023

(54) OCTENIDINE SALT ANTIMICROBIAL MEDICAL ARTICLES

(71) Applicant: 3M INNOVATIVE PROPERTIES COMPANY, St. Paul, MN (US)

(72) Inventors: Vinod P. MENON, Woodbury, MN (US); Debra L. SCHWAB, Stillwater, MN (US)

(21) Appl. No.: 17/789,262

(22) PCT Filed: Dec. 22, 2020
(86) PCT No.: PCT/IB2020/062343

§ 371 Date: Jun. 27, 2022

Related U.S. Application Data
(60) Provisional application No. 62/955,937, filed on Dec. 31, 2019.

(51) Int. Cl. A61L 31/16; A01N 25/02; A01N 25/08; A01N 43/14

(52) U.S. Cl. CPC A61L 31/16; A01N 25/02; A01N 25/08; A01N 43/14; A61L 2300/204; A61L 2300/404

(57) ABSTRACT

Homogenous octenidine solutions and compositions are described. Also described are medical articles (e.g., incise drapes) including octenidine formulations and methods for preparing the same.

### BACKGROUND

Surgical-site infections often lead to prolonged hospitalizations and are heavily associated with high morbidity and mortality. A patient's risk for surgical-site infection is largely proportional to the residual bacteria at the wound site. The steady increase in antibiotic resistance makes pre-operative efforts to reduce bacteria density at surgical sites crucial.

Surgical incise drapes having antimicrobial-impregnated adhesives are an effective way to keep surgical sites from recolonizing after initial preparation and during operation. The only approved antimicrobial drapes are iodophor-based. However, some percentage of the population is sensitive or allergic to iodophor antimicrobials. For at least this reason, surgical drapes having different antimicrobial are needed.

Octenidine hydrochloride has been used since the late 1980s as an active antiseptic against Gram-positive and Gram-negative bacteria. Ideally, a formulation including an octenidine salt in a hydrophobic medium would allow for suitable drape formulations. Unfortunately, octenidine salts are not readily soluble in typical hydrophobic mediums.

What is needed is a way to formulate octenidine salts alongside pressure-sensitive adhesives for use in surgical drapes. The present disclosure demonstrates that octenidine salts may be surprisingly incorporated in hydrophobic solubilizers that have a proximate pair of hydrogen-bonding groups.

### SUMMARY

In one embodiment, a homogenous solution is described. The homogenous solution may include a solubilizer having a pair of vicinal hydrogen-bonding groups, wherein at least one hydrogen-bonding group is a hydrogen-bond donor and having a saturated or unsaturated C₇-C₂₂ hydrocarbon group. The homogenous solution may include an octenidine salt present in an amount greater than 0 wt % and up to about 20 wt % with respect to the weight of the solubilizer. The octenidine salt may be solubilized in the solubilizer to provide the homogenous solution at a temperature of about 20-25° C., or at a temperature equivalent to the melting point temperature of the solubilizer. The homogenous solution may further include water present in an amount less than about 5 wt % with respect to the weight of the homogenous solution. The homogenous solution may include a hydrophilic vehicle described herein present in amount in mols less than about 2:1 with respect to the amount of octenidine salt.

In one embodiment, a composition is described. The composition may include a one or more solubilizer having a pair of vicinal hydrogen-bonding groups, wherein one hydrogen-bonding group is a hydrogen-bond donor and having a saturated or unsaturated C₇₋₂₂ hydrocarbon group. The composition may include an octenidine salt present in an amount greater than 0 wt % and up to about 20 wt % with respect to the weight of the one or more solubilizer. The composition may include water present in an amount less about 5 wt % with respect to the weight of the solubilizer and octenidine salt combined. The composition may include a hydrophilic vehicle present in an amount in mols of less than about 2:1 with respect to the octenidine salt. The composition may include a pressure-sensitive adhesive and a plasticizer.

In one embodiment, a medical article is described. The medical article may include any octenidine-containing composition described herein and a drape backing.

In one embodiment, a method for preparing a homogenous solution is described. The method may include providing an octenidine salt described herein and one or more solubilizer described herein. The method may include blending the octenidine salt and the one or more solubilizer to form a homogenous solution.

In one embodiment, a method for preparing a composition is described. The method may include providing an octenidine salt described herein; a solubilizer described herein; a pressure-sensitive adhesive described herein; and a plasticizer described herein. The method may include blending the octenidine salt, the solubilizer, the pressure-sensitive adhesive, and the plasticizer to form the composition.

In one embodiment, a method for preparing a medical article is described. The method may include providing an octenidine salt described herein; a solubilizer described herein; a pressure-sensitive adhesive described herein; and a plasticizer described herein. The method may include blending the octenidine salt, the solubilizer, the pressure-sensitive adhesive, and the plasticizer to form a composition described herein. The method may include coating a release liner with the composition to form a wet-coated release liner and drying the wet-coated release liner under a set of conditions to form a dry-coated release liner. The method may include laminating the dry-coated release liner to a film to form the medical article.

### DETAILED DESCRIPTION

Octenidine dihydrochloride is rapidly gaining acceptance as a broad-spectrum antimicrobial, and is being used for disinfection, antisepsis, and decolonization in health-care settings. Octenidine is not absorbed through skin or mucous membranes and does not cause contact dermatitis. No reports have demonstrated reduced susceptibility to octenidine. Furthermore, octenidine has no apparent toxicity, unlike other cationic biocides, and has been shown to be less cytotoxic than chlorhexidine. In fact, the antimicrobial efficacy for octenidine is about 3-10 times higher than chlorhexidine.

The present disclosure provides compositions for effectively delivering octenidine to and from pressure-sensitive adhesives. Glycol monoesters, which include only one hydroxyl group, are demonstrated as suitable solvents for octenidine.

As used herein, “alkanol” refers to an alkyl (i.e., aliphatic) alcohol.

As used herein, “alkyl” means a linear or branched hydrocarbon chain. The hydrocarbon chain is saturated unless indicated otherwise. For example, a C₁₋₆ alkyl means a C₁ alkyl (i.e., methyl), a C₂ alkyl (i.e., ethyl), C₃ alkyl (i.e., propyl, isopropyl), C₄ alkyl (e.g., butyl, isobutyl, secbutyl), C₅ alkyl (e.g., pentyl), or C₆ (e.g., hexyl).

As used herein, “alkenyl” means a linear or branched hydrocarbon chain having one or more units of unsaturation of the formula: (R)₂C═C(R)₂, wherein each R is arbitrary.

As used herein, “alkynyl” means a linear or branched hydrocarbon chain having one or more units of unsaturation of the formula: R—C≡C—R, wherein each R is arbitrary.

As used herein, “antimicrobial” refers to antiseptic. The terms are used interchangeably herein.

As used herein, “alkaryl” refers to an alkylene and aryl combined, wherein the alkylene is the point of attachment. For example, X—R where R is an alkaryl means X-alkylene-aryl.

As used herein, “aralkyl” refers to an aryl and alkyl combined, wherein the aryl group is the point of attachment. For example, X—R where R is an aralkyl means X-aryl-alkyl.

As used herein, “aryl” describes a heteroatom-free aromatic moiety. An aromatic compound is cyclic, planar, fully conjugated, and follows Hückles Rule (i.e., having 4n+2 π-electrons, wherein n is an integer). For example, phenyl is a C₆ aryl, naphthyl and azulenyl are C₁₀ aryls, and anthracenyl and phenanthrenyl are C₁₄ aryls. An aryl group may be unsubstituted, or substituted with groups such as C₁₋₆ alkyl optionally substituted with one or more halogens (e.g., —CF₃), C₁₋₆ alkoxy (e.g., —OCH₃, —OCH₂CH₃, or the like) optionally substituted with one or more halogens (e.g., —OCF₃), halogens (e.g., I, Br, Cl, F), or the like.

As used herein, “about” means ±10 percent of a given value. For example, about 10 means 9 to 11.

As used herein, “C_{X}-C_{Y}” or “C_{X-Y”}” prefacing a chemical moiety name refers to the number of carbon atoms in said moiety, wherein X and Y are integers. For example, a C₃₋₆ cycloalkyl refers to, for example, cyclopropyl (C3), cyclobutyl (C4), cyclopentyl (C5), or cyclohexyl (C6).

As used herein, “carbonyl moiety” means —C(O)—, which is equivalent to —C(═O)—.

As used herein, “(cyclo)alkyl” refers to a moiety that describes a cycloalkyl group or an alkyl group. A cycloalkyl group is a cyclic, bicyclic, fused or bridged bicyclic alkyl group, e.g., cyclopropyl, cyclobutyl, cyclopentyl, cyclohexyl, isobornyl, or the like.

As used herein, “fatty” or “fatty unit” refers to the C₇-C₂₂ hydrocarbon moiety described herein.

As used herein, “glycol” describes a compound or moiety having two alcohols separated by two carbons.

As used herein, “glycol fatty monoester” describes a compound having one or more glycol unit wherein at least one alcohol forms a bond to the compound via an ester linkage. A glycol fatty monoester has at least one glycol unit having two oxygen groups separated by no more than two carbons wherein one oxygen group is —OH. Unless indicated otherwise, specific compound names such as propylene glycol monoheptanoate means one propylene glycol unit. The pre-fix “poly” will be used to indicate more than one glycol unit, e.g., polypropylene glycol monoheptanoate. Likewise, “glycol fatty monoester” describes a compound having one or more glycol unit wherein at least one alcohol forms a bond to the compound via an ether linkage.

As used herein, “glycerol” refers to a compound of the formula: HO—CH₂—CH(OH)—CH₂—OH.

As used herein, “glycerol fatty mono- or di-ester” describes a compound having one or more glycerol unit wherein at least one alcohol forms a bond to the compound via an ether linkage. Unless indicated otherwise, specific compound names such as glycerol monoisostearate means one glycerol unit. The pre-fix “poly” will be used to indicate more than one glycerol unit, e.g., polyglycerol monoisostearate. Likewise, “glycol fatty monoether” describes a compound having one or more glycol unit wherein at least one alcohol forms a bond to the compound via an ether linkage.

As used herein, “hydrocarbon group” refers to a group consisting of carbon and hydrogen. A C₇-C₂₂ hydrocarbon group refers to a group having 7-22 carbons in a linear chain. The linear chain may include cyclic groups, i.e., (cyclo)alkyl. For example, a butylbenzyl group includes seven carbons in the longest linear carbon chain. The term “saturated hydrocarbon group” refers to groups without alkenyl, alkynyl, or aryl groups. The term “unsaturated hydrocarbon group” refers to groups having one or more alkenyl, alkynyl, and aryl groups.

As used herein, “heteroaryl” describes a moiety including at least one heteroatom-containing aromatic group. Heteroaryls described include 1-4 heteroatoms selected from nitrogen, oxygen, and sulfur. An aromatic compound is cyclic, planar, fully conjugated, and follows Hückles Rule (i.e., having 4n+2 π-electrons, wherein n is an integer). A heteroaryl group may include non-aromatic cyclic groups fused to the heteroaryl. A heteroaryl group may be unsubstituted, or substituted with groups such as C₁₋₆ alkyl optionally substituted with one or more halogens (e.g., —CF₃), C₁₋₆ alkoxy (e.g., —OCH₃, —OCH₂CH₃, or the like) optionally substituted with one or more halogens (e.g., —OCF₃), halogens (e.g., I, Br, Cl, F), or the like.

As used herein, “homogenous solution” refers to a visually clear or transparent solution. No particulates or cloudiness is observed in the homogeous solutions described herein. A homogenous solution excludes dispersions, suspension, and the like.

As used herein, “hydrogen-bond donor” refers to a chemical moiety of the formula —ZH, wherein H is hydrogen and Z is an atom that has an electronegativity value greater than the electronegativity value of hydrogen (i.e., 2.2), and is not carbon. As used herein, “hydrogen-bond acceptor” refers to a chemical moiety having an atom with a lone pair of electrons, wherein the atom has an electronegativity value greater than the electronegativity value of hydrogen (i.e., 2.2).

As used herein, “hydrophilic-lipophilic balance” or “HLB” values are calculated using the method of Griffin (Griffin W C; J. Soc. of Cosmetic Chemists 5, 259 (1954)). “The HLB Method” used herein involves a calculation based on the following:

HLB=(E+P)/5

wherein E is the weight percent of oxyethylene content and P is the weight percent of polyhydric alcohol content (e.g., glycerol, sorbitol, etc.). For the compounds herein, glycerol segments with two hydroxyl groups, glycerol segments with one hydroxyl group, and hydroxyl-containing segments of any additional polyhydric molecules were included in the definition of P.

As used herein, “hydrophilic vehicle” generally describes a compound having an HLB greater than 10. The term “hydrophilic vehicle” is not intended to include water. Water is treated as its own entity herein.

As used herein, “solubilizer” generally describes a compound having an HLB no greater than 10. A hydrophobic vehicle may have an HLB value of no greater than 10, 9, 8, 7, 6, 5, 4, 3, 2, or 1. As used herein, “hydrophobic vehicle” may describe a “solubilizer” or a “plasticizer.” Use of the term “hydrophobic vehicle” is reserved for components

As used herein, “hydroxyalkyl” refers to a hydroxy-substituted alkyl group, e.g., —CH₂CH₂OH, —CH(OH)CH₂(OH), or the like.

As used herein, “(meth)acrylate” refers to a moiety that describes a methacrylate group, e.g., ROC(O)C(CH₃)═CR₂, or an acrylate group, e.g., ROC(O)CH═CR₂, wherein in each R is arbitrary. Likewise, “(meth)acrylamide” refers to a moiety that describes a methacrylamide group, e.g., R₂NC(O)C(CH₃)═CR₂, or an acrylamide group, e.g., R₂NC(O)CH═CR₂, wherein in each R is arbitrary.

As used herein, “mono” means one. For example, a “monoester” means a compound having one ester. As used herein, “di” means two. For example, a “diester” means a compound having two esters.

As used herein, “optionally substituted” described a group that may or may not be substituted with a given substituent. The substitution reflects the exchange of a hydrogen for the given substituent. For example, an alkyl group, e.g., —CH₂CH₂CH₃ may or may not be (i.e., optionally) substituted with a hydroxyl group, e.g., —CH(OH)CH₂CH₃, —CH₂CH(OH)CH₃, or —CH₂CH₂CH₂OH. For example, a hydroxylalkyl group, e.g., —CH₂CH₂CH₂OH, may or may not be (i.e., optionally) substituted with a phenyl group, e.g., —CH(Ph)CH₂CH₂OH, —CH₂CH(Ph)CH₂OH, —CH₂CH₂CH(Ph)OH, or —CH₂CH₂CH₂O-Ph.

As used herein, “plasticizer” describes an agent that decreases the glass transition temperature of a material.

As used herein, “preventing” or “prevent” describes reducing or eliminating the onset of the symptoms or complications of a disease, condition, or disorder that is caused or aggravated by an infection.

As used herein, a “salt” is used interchangeably with “pharmaceutically acceptable salt” or pharmaceutically acceptable counterion”, e.g., anion, and refers to those salts of the compounds formed by the process of the present application which are, within the scope of sound medical judgment, suitable for use in contact with the tissues of humans and animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the application, or separately by reacting the free base or acid function with a suitable acid or base.

As used herein, “saturated” described a hydrocarbon that does not include one or more olefins, i.e., alkenyl or alkynyl. Conversely, “unsaturated” or “unsaturation” describes a hydrocarbon that includes one or more olefins, i.e., alkenyl or alkynyl, represented by the formulae, respectively: (R)₂C═C(R)₂ or R—C≡C—R, wherein each R is arbitrary.

As used herein, “subject” refers to a mammal. A subject therefore refers to, for example, dogs, cats, horses, cows, pigs, guinea pigs, or the like. Preferably the subject is a human. When the subject is a human, the subject may be referred to herein as a patient.

As used herein, “vicinal” describes the relationship between two groups that are bonded to adjacent carbons. For example, X and Y are vicinal, i.e., bonded to adjacent carbons, in the following formulae: H₃C—CH(Y)—CH(X)—CH₃, H₃C—CH(Y)—CH₂—X—CH₃, or H₃C—Y—CH₂CH₂—X—CH₃.

As used herein, “proximate” describes the relationship between two groups, e.g., heteroatoms, that are separated by 2-3 bonds. Groups that are separated by 2 bonds are bonded to the same carbon atom. For example, O═ and —NH— groups of a sulfonamide, e.g., R—S(O)₂NH—R, are separated by 2 bonds. Groups that are separated by 3 bonds are bonded to adjacent carbon atoms. As used herein, “vicinal” describes the proximate relationship between two groups that are separated by 3 bonds. For example, the heteroatoms of the following are considered vicinal: —CH(OH)CH(OH)—, —OCH₂CH₂OH, —NHCH₂CH₂OH, or the like.

As used herein, “tackifier” refers to an agent that increases the glass transition temperature of a material.

### Octenidine Solutions

In various embodiments, a homogenous solution is described. The homogenous solution may include a solubilizer described herein, the solubilizer having a pair of vicinal hydrogen-bonding groups, wherein at least one hydrogen-bonding group is a hydrogen-bond donor and having a saturated or unsaturated C₇-C₂₂ hydrocarbon group. The homogenous solution may include an octenidine salt described herein that is present in an amount greater than 0 wt % and up to about 20 wt % with respect to the weight of the solubilizer. The octenidine salt may be solubilized in the solubilizer to provide the homogenous solution at a temperature of about 20-25° C., or at a temperature equivalent to the melting point temperature of the solubilizer. The homogenous solution may further include water present in an amount less than about 5 wt % with respect to the weight of the homogenous solution. The homogenous solution may include a hydrophilic vehicle described herein present in amount in mols less than about 2:1 with respect to the amount of octenidine salt.

In some embodiments, the homogenous solution may consist essentially of the solubilizer and the octenidine salt.

In some embodiments, the pair of vicinal hydrogen-bonding groups may include one hydrogen-bond donor and one hydrogen-bond acceptor.

In some embodiments, the pair of vicinal hydrogen-bonding groups may each be hydrogen-bond donors.

In some embodiments, the homogenous solution may include water present in amount in wt % with respect to the weight of the homogenous solution of less than about 5, less than about 4, less than about 3, less than about 2, less than about 1, less than about 0.5, less than about 0.1, or a value within a range between any of the preceding values, for example, between about 1 wt % and about 2 wt %, between about 0.5 wt % and about 3 wt %, or the like. In some embodiments, the homogenous solution is free of water, i.e., less than 0.001 wt %.

In some embodiments, the homogenous solution may include a hydrophilic vehicle present in an amount in mol with respect to 1.0 mol octenidine salt of about 2, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1, or a value between any of the preceding values, for example, between about 1.5 and about 1.0, between about 1.8 and about 0.4, or the like. In some embodiments, a hydrophilic vehicle may be present in mol of less than 0.1:1 octenidine salt.

### Octenidine Drape Compositions

In various embodiments, a composition is described. The composition may include a one or more solubilizer described herein, the one or more solubilizer having a pair of vicinal hydrogen-bonding groups, wherein one hydrogen-bonding group is a hydrogen-bond donor and having a saturated or unsaturated C₇₋₂₂ hydrocarbon group. The composition may include an octenidine salt described herein in an amount greater than 0 wt % and up to about 20 wt % with respect to the weight of the one or more solubilizer. The composition may include water present in an amount less about 5 wt % with respect to the weight of the solubilizer and octenidine salt combined. The composition may include a hydrophilic vehicle present in an amount in mols of less than about 2:1 with respect to the octenidine salt. The composition may include a pressure-sensitive adhesive and a plasticizer.

In some embodiments, the one or more solubilizer may be present in an amount between about 1 wt % and about 15 wt % with respect to the weight of the composition. The one or more solubilizer may be present in an amount in wt % of about 1, 2, 3, 4, 5, 6, 7, 8, 9, 10, 11, 12, 13, 14, or 15 or a value within a range between any of the preceding values, for example, between about 5 and about 10, between about 8 and about 12, or the like.

In many embodiments, the octenidine salt may be present in an amount between about greater than 0 wt % to about 20 wt % with respect to the weight of the solubilizer. For example, the octenidine salt may be present in amount in wt % with respect to the solubilizer of about 0.01, 0.05, 0.1, 0.5, 1.0, 2.0, 3.0, 4.0, 5.0, 6.0, 8.0, 10.0, 12.0, 14.0, 16.0, 18.0, or 20.0, or a value within a range between any of the preceding values, for example, between about 1.0 and about 5.0, between about 5.0, and about 12.0, or the like. In many embodiments, the octenidine salt and one or more solubilizer form a homogenous solution described herein.

In many embodiments, the octenidine salt may be present in an amount between about 0.01 and about 2 wt % with respect to the weight of the composition. For example, the octenidine salt may be present in wt % with respect to the weight of the composition of about 0.01, 0.02, 0.04, 0.06, 0.08, 0.10, 0.12, 0.14, 0.16, 0.18, 0.20, 0.22, 0.24, 0.26, 0.28, 0.30, 0.35, 0.40, 0.45, 0.50, 0.55, 0.60, 0.65, 0.70, 0.75, 0.80, 0.85, 0.90, 0.95, 1.0, 1.1, 1.2, 1.3, 1.4, 1.5, 1.6, 1.7, 1.8, 1.9, or 2.0, or a value within a range between any of the preceding values, for example, between about 0.16 and about 0.24, between about 1.0 and about 1.3, or the like.

In some embodiments, the composition may include water present in amount in wt % with respect to the weight of the solubilizer and octenidine salt combined of less than about 5, less than about 4, less than about 3, less than about 2, less than about 1, less than about 0.5, less than about 0.1, or a value within a range between any of the preceding values, for example, between about 1 wt % and about 2 wt %, between about 0.5 wt % and about 3 wt %, or the like. In some embodiments, the composition is free of water, i.e., less than 0.001 wt %.

In some embodiments, the composition may include a hydrophilic vehicle present in an amount in mol with respect to 1.0 mol octenidine salt of about 2, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1, or a value between any of the preceding values, for example, between about 1.5 and about 1.0, between about 1.8 and about 0.4, or the like. In some embodiments, a hydrophilic vehicle may be present in mol of less than 0.1:1 octenidine salt.

In some embodiments, the pressure-sensitive adhesive may be present in amount of between about 55 wt % to about 85 wt % with respect to the weight of the composition. The pressure-sensitive adhesive may be present in an amount in wt % with respect to the weight of the composition of about 55, 60, 65, 66, 67, 68, 69, 70, 71, 72, 73, 74, 75, 80, or 85, or a value within a range between of any of the preceding values, for example, between about 65 and about 75, between about 70 and about 80, or the like.

In many embodiments, the octenidine salt and the plasticizer together do not form a homogenous solution at a temperature of about 20-25° C. or higher. In other words, suitable plasticizers are not capable of solubilizing an octenidine salt.

In some embodiments, the plasticizer may be present in amount of between about 10 wt % to about 30 wt % with respect to the weight of the composition. The plasticizer may be present in an amount in wt % with respect to the weight of the composition of about 10, 12, 14, 16, 18, 20, 22, 24, 26, 28, or 30, or a value within a range between any of the preceding values, for example, between about 15 and about 25, between about 18 and about 22, or the like.

In some embodiments, the composition may include, in wt % with respect to the weight of the composition, the one or more solubilizer present in an amount of between about 5 wt % and about 10 wt %, the octenidine salt present in amount up to about 2 wt %, the pressure-sensitive adhesive present in an amount of about 65 wt % to about 75 wt %, and the plasticizer present in an amount of about 15 wt % to about 25 wt %.

### Octenidine Salt

In some embodiments, the octenidine salt may be present in an amount in wt % with respect to the weight of the solubilizer of about 0.1, 0.5, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, or 20.0, or a value in a range between any of the preceding values, for example, between about 5.0 and about 10.0, between about 8.0 and about 12.0, or the like.

In some embodiments, the octenidine salt may include any pharmaceutically-acceptable anions, for example, anions selected from chloride, bromide, iodine, sulfate, phosphate, phosphite, nitrite, nitrate, or the like. Suitable pharmaceutically acceptable salts may be found in J. Pharmaceutical Sciences 1977, 66, 1-19, which is incorporated herein by reference in its entirety.

In several embodiments, the octenidine salt is octenidine hydrochloride.

### Solubilizer

In some embodiments, any solubilizer described herein may be characterized by having a hydrophilic-lipophilic balance of no greater than 10 as determined using the HLB Method.

In some embodiments, the vicinal hydrogen-bonding groups may independently be selected from hydroxyl (—OH), oxy (—O—), or amino (—N(R¹)(R²) or —N(R³)—). In some embodiments, R¹, R², and R³ are independently selected from hydrogen (—H), C₁₋₆ alkyl (e.g., —CH₃), a 2-hydroxy C₂-C₆ alkyl group (e.g., —CH₂CH₂OH), and a 2-amino C₂-C₆ alkyl group (e.g., —CH₂CH₂N(R^{1′})(R^{2′}), wherein R^{1′} and R^{2′} are independently selected from hydrogen (—H) and C₁₋₆ alkyl).

In some embodiments, the vicinal groups include —OH and —OH.

In some embodiments, the vicinal groups include —OH and —O—.

In some embodiments, the vicinal groups include —OH and —N(R³)—. In some embodiments, R³ is —H. In other embodiments, R³ is C₁ alkyl. In other embodiments, R³ is —CH₂CH₂OH.

In some embodiments, the vicinal groups include —N(R¹)(R²) and —O—. In some embodiments, at least one of R¹ and R² is —H. In some embodiments, each of R¹ and R² is —H. In other embodiments, R¹ is —H and R² is C₁₋₆ alkyl, e.g., C₁ alkyl. In some embodiments, one or more of R¹ and R² is independently a 2-hydroxy C₂-C₆ alkyl group or 2-amino C₂-C₆ alkyl group.

In some embodiments, the vicinal groups include —N(R¹)(R²) and —N(R³)—. In some embodiments, at least one of R¹ and R² is —H. In some embodiments, each of R¹ and R² is —H. In other embodiments, R¹ is —H and R² is C₁₋₆ alkyl, e.g., C₁ alkyl. In some embodiments, R³ is —H. In other embodiments, R³ is C₁₋₆ alkyl, e.g., C₁ alkyl. In some embodiments, at least one R¹ and R² is —H and R³ is C₁₋₆ alkyl, e.g., C₁ alkyl. In other embodiments, each of R¹ and R² are independently C₁₋₆ alkyl and R³ is —H. In some embodiments, one or more of R¹, R², and R³ is independently a 2-hydroxy C₂-C₆ alkyl group or 2-amino C₂-C₆ alkyl group.

In many embodiments, the solubilizer may further include one or more carbonyl (i.e., C═O). In some embodiments, the carbonyl moiety is an amide (i.e., —N(R³)—C(O)—). In some embodiments, the carbonyl moiety is an ester (i.e., —O—C(O)—). In some embodiments the carbonyl moiety is a carbonate (i.e., —O—C(O)—O—). In some embodiments the carbonyl moiety is a carbamate (i.e., —N(R³)—C(O)—O—). In some embodiments the carbonyl moiety is a urea (i.e., —N(R³)—C(O)—N(R³)—).

In some embodiments, the C₇-C₂₂ hydrocarbon group is a C₇-C₂₂ alkyl group. In some embodiments, the C₇-C₂₂ hydrocarbon group is selected from fatty units of caprylic acid (C8), capric acid (C10), lauric acid (C12), myristic acid (C14), palmitic acid (C16), stearic acid (C18), arachidic acid (C20), and behenic acid (C22).

In some embodiments, the C₇₋₂₂ hydrocarbon group is a C₇-C₂₂ alkyl group including one or more hydroxyl (—OH) groups. For example, the C₇₋₂₂ hydrocarbon group may be derived from 16-hydroxyhexadecanoic acid.

In some embodiments, the C₇₋₂₂ hydrocarbon group is an C₇-C₂₂ alkenyl group having one or more units of unsaturation. In some embodiments, the C₇-C₂₂ alkenyl group may include one unit of unsaturation. In other embodiments, the C₇-C₂₂ alkenyl group may include two units of unsaturation. In other embodiments, the C₇-C₂₂ alkenyl group may include three units of unsaturation. In some embodiments, the C₇-C₂₂ alkenyl group may include up to six units of unsaturation. One or more units of unsaturation may be cis in orientation. In some embodiments, all units of unsaturation may be cis in orientation. In some embodiments, one or more units of unsaturation may be trans in orientation.

In some embodiments, the C₇₋₂₂ hydrocarbon group may be selected from fatty units of dodecenoic acid (C12), tetradecenoic acid (C14), palmitoleic acid (C16), vaccenic acid (C18), rumenic acid (C18), paullinic acid (C20), and docosenoic acid (C22).

In some embodiments, the C₇₋₂₂ hydrocarbon group may be selected from fatty units of hypogeic acid (C16), oleic acid (C18), elaidic acid (C18), gondoic acid (C20), mead acid (C20), erucic acid and (C22).

In some embodiments, the C₇₋₂₂ hydrocarbon group may be selected from fatty units of hexadecatrienoic acid (C16), alpha-linolenic acid (C18), stearidonic acid (C18), eicosatrienoic acid (C20), Eicosatetraenoic acid (C20), eicosapentaenoic acid (C20), heneicosapentaenoic acid (C21), docosapentaenoic acid (C22), and docosahexaenoic acid (C22).

In some embodiments, the C₇₋₂₂ hydrocarbon group may be selected from fatty units of linoleic acid (C18), gamma-linolenic acid (C18), calendic acid (C18), eicosadienoic acid (C20), dihomo-gamma-linolenic acid (C20), arachidonic acid (C20), docosadienoic acid (C22), adrenic acid (C22), and osbond acid (C22).

In some embodiments, the C₇₋₂₂ hydrocarbon group is a C₇-C₂₂ alkenyl group including one or more hydroxyl (—OH) groups.

In some embodiments, the solubilizer may be represented by the formula:

R^{a}—W—R^{b},

wherein:
R^{a} is an C₂₋₆ alkyl, C₆₋₁₂ aralkyl, C₆₋₁₀ alkaryl, or C₆₋₁₀ aryl, wherein the C₂₋₆ alkyl, C₆₋₁₂ aralkyl, C₆₋₁₀ alkaryl, or C₆₋₁₀ aryl is substituted with one or more of —OH and —N(R¹)(R²), and is optionally substituted with —W—R^{b};
each W is independently selected from —O—, —N(R³)—, —O—C(O)—, —C(O)—O—, —O—C(O)—O—, —O—C(O)—N(R³)—, —N(R³)—C(O)—O—, —N(R³)—C(O)—, —C(O)—N(R³)—, or —N(R³)—C(O)— N(R³)—;
R^{b} is a C₇-C₂₂ hydrocarbon group selected from C₇₋₂₂ alkyl and C₇₋₂₂ alkenyl, wherein the C₇₋₂₂ alkyl and C₇₋₂₂ alkenyl are optionally substituted with one or more hydroxyl;
R¹, R², and R³ are independently selected from —H, C₁₋₆ alkyl, 2-hydroxy C₂-C₆ alkyl, 2-amino C₂-C₆ alkyl.

In some embodiments, the solubilizer is represented by formula (I) and R^{a} is selected from HO—(CH₂CH₂O)ₘ—CH₂CH₂—, HO—(CH(CH₃)CH₂O)ₘ—CH(CH₃)CH₂—, and HO—(CH₂CH(CH₃)O)ₘ—CH₂CH(CH₃)—. m may be an integer selected from 0-3.

In some embodiments, the solubilizer is represented by formula (I) and R^{a} is HO—CH₂CH₂—.

In some embodiments, the solubilizer is represented by formula (I) and R^{a} is HO—CH(CH₃)CH₂—.

In some embodiments, the solubilizer is represented by formula (I) and R^{a} is HO—CH₂CH(CH₃)—.

In some embodiments, the solubilizer is represented by formula (I) and R^{a} is HO—CH₂CH(OH)—CH₂—.

In some embodiments, the solubilizer is represented by formula (I) and R^{a} is —O—C(O)—.

In some embodiments, the solubilizer is represented by formula (I) and R^{a} is —N(R³)—C(O)—.

In some embodiments, the solubilizer is represented by formula (I), R^{a} is —N(R³)—C(O)—, and R³ is —H.

In some embodiments, the solubilizer is represented by formula (I), R^{a} is —N(R³)—C(O)—, and R³ is —CH₃.

In some embodiments, the solubilizer is represented by formula (I), R^{a} is —N(R³)—C(O)—, and R³ is —CH₂CH₂OH.

In some embodiments, the solubilizer is represented by formula (I), R^{a} is —N(R³)—C(O)—, and R³ is —CH₂CH(OH)CH₂OH.

In some embodiment, the solubilizer is represented by formula (I) and R^{b} is a C₇₋₈ alkyl group.

In some embodiments, the solubilizer is represented by formula (I) and R^{b} is a C₁₁₋₁₂ alkyl group.

In some embodiments, the solubilizer is represented by formula (I) and R^{b} is a C₁₇₋₁₈ alkyl group.

In some embodiments, the solubilizer is represented by formula (I), R^{a} is HO—CH₂CH₂—and W is —O—C(O)—.

In some embodiments, the solubilizer is represented by formula (I), R^{a} is HO—CH(CH₃)CH₂— and W is —O—C(O)—.

In some embodiments, the solubilizer is represented by formula (I), R^{a} is HO—CH₂CH(CH₃)— and W is —O—C(O)—.

In some embodiments, the solubilizer is represented by formula (I) R^{a} is HO—CH₂CH(OH)—CH₂— and W is —O—C(O)—.

In some embodiments, the solubilizer is represented by formula (I), R^{a} is HO—CH₂CH₂—, W is —N(R³)—C(O)—, and R³ is —H.

In some embodiments, the solubilizer is represented by formula (I), R^{a} is HO—CH(CH₃)CH₂— or HO—CH₂CH(CH₃)—, W is —N(R³)—C(O)—, and R³ is —H.

In some embodiments, the solubilizer is represented by formula (I) R^{a} is HO—CH₂CH(OH)—CH₂—, W is —N(R³)—C(O)—, and R³ is —H.

In some embodiments, the solubilizer is represented by formula (I), R^{a} is HO—CH₂CH₂—, W is —N(R³)—C(O)—, and R³ is —CH₃.

In some embodiments, the solubilizer is represented by formula (I), R^{a} is HO—CH(CH₃)CH₂— or HO—CH₂CH(CH₃)—, W is —N(R³)—C(O)—, and R³ is —CH₃.

In some embodiments, the solubilizer is represented by formula (I) R^{a} is HO—CH₂CH(OH)—CH₂—, W is —N(R³)—C(O)—, and R³ is —CH₃.

In some embodiments, the solubilizer is represented by formula (I), R^{a} is HO—CH₂CH₂—, W is —N(R³)—C(O)—, and R³ is —CH₂CH₂OH.

In some embodiments, the solubilizer is represented by formula (I), R^{a} is HO—CH(CH₃)CH₂— or HO—CH₂CH(CH₃)—, W is —N(R³)—C(O)—, and R³ is —CH₂CH₂OH.

In some embodiments, the solubilizer is represented by formula (I), R^{a} is HO—CH(CH₃)CH₂— or HO—CH₂CH(CH₃)—, W is —N(R³)—C(O)—, and R³ is —CH(CH₃)CH₂OH or —CH₂CH(CH₃)OH.

In some embodiments, the solubilizer is represented by formula (I) R^{a} is HO—CH₂CH(OH)—CH₂—, W is —N(R³)—C(O)—, and R³ is —CH₂CH₂OH.

In some embodiments, the solubilizer is represented by formula (I) R^{a} is HO—CH₂CH(OH)—CH₂—, W is —N(R³)—C(O)—, and R³ is —CH(CH₃)CH₂OH or —CH₂CH(CH₃)OH.

In some embodiments, the solubilizer is represented by formula (I) R^{a} is HO—CH₂CH(OH)—CH₂—, W is —N(R³)—C(O)—, and R³ is —CH₂CH(OH)CH₂OH.

In some embodiments, the solubilizer may be represented by formula (II):

R^{b}—C(O)O—R^{a}—O—C(O)—R^{b} (II),

wherein:

R^{a} is an C₂₋₆ alkyl, C₆₋₁₂ aralkyl, C₆₋₁₀ alkaryl, or C₆₋₁₀ aryl, wherein the C₂₋₆ alkyl, C₆₋₁₂ aralkyl, C₆₋₁₀ alkaryl, or C₆₋₁₀ aryl is substituted with one or more of —OH and —N(R¹)(R²), and

R^{b} is a C₇-C₂₂ hydrocarbon group selected from C₇₋₂₂ alkyl and C₇₋₂₂ alkenyl, wherein the C₇₋₂₂ alkyl and C₇₋₂₂ alkenyl are optionally substituted with one or more hydroxyl;

In some embodiments, the solubilizer is represented by formula (II) and R^{a} is selected from —CH₂CH(OH)CH₂— and —CH(CH₂OH)CH₂—.

In some embodiment, the solubilizer is represented by formula (I) and R^{b} is a C₇₋₈ alkyl group.

In some embodiments, the solubilizer is represented by formula (II) and R^{b} is a C₁₁₋₁₂ alkyl group.

In some embodiments, the solubilizer is represented by formula (II) and R^{b} is a C₁₇₋₁₈ alkyl group.

In some embodiments, the solubilizer is represented by one of the following formulae: HO—CH₂CH₂—O—C(O)—R^{b}, HO—CH(CH₃)CH₂—O—C(O)—R^{b}, HO—CH₂CH₂(OH)CH₂—O—C(O)—R^{b}, R^{b}—C—(O)—O—CH₂CH₂(OH)CH₂—O—C(O)—R^{b}, HO—CH₂CH₂(O—C(O)—R^{b})CH₂—O—C(O)—R^{b}, HO—CH₂CH₂—NH—C(O)—R^{b}, HO—CH(CH₃)CH₂—NH—C(O)—R^{b}, HO—CH₂CH₂—N(CH₂CH₂OH)—C(O)—R^{b}, and HO—CH(CH₃)CH₂—N(CH₂(CH₃)CH—OH)—C(O)—R^{b},

wherein R^{b} is a C₇-C₂₂ hydrocarbon group selected from C₇₋₂₂ alkyl and C₇₋₂₂ alkenyl, wherein the C₇₋₂₂ alkyl and C₇₋₂₂ alkenyl are optionally substituted with one or more hydroxyl.

In some embodiments, the solubilizer is selected from a glycol fatty monoester, an ethanolamine fatty monoester, an ethanolamine fatty monoamide, an ethylenediamine fatty monoamide, a glycerol fatty monoester, a glycerol fatty diester, an aminopropanediol fatty monoester, an aminopropanediol fatty monoamide, a diaminopropanol fatty monoamide, and a diaminopropanol fatty monoester.

In some embodiments, the solubilizer has one or more fatty units independently selected from caprylate, caprate, laurate, myristate, palmitate, oleate, stearate, and isostearate.

In some embodiments, the solubilizer is a glycol fatty monoester selected from propylene glycol monocaprylate, propylene glycol monocaprate, propylene glycol monoheptanoate, propylene glycol monolaurate, propylene glycol monomyristate, propylene glycol monopalmitate, propylene glycol monooleate, propylene glycol monostearate, and propylene glycol monoisostearate.

In some embodiments, the solubilizer is a propylene glycol fatty monoester.

In some embodiments, the solubilizer is a glycol fatty monoester selected from ethylene glycol monocaprylate, ethylene glycol monocaprate, ethylene glycol monoheptanoate, ethylene glycol monolaurate, ethylene glycol monomyristate, ethylene glycol monopalmitate, ethylene glycol monooleate, ethylene glycol monostearate, and ethylene glycol monoisostearate.

In some embodiments, the solubilizer is an ethylene glycol fatty monoester.

In some embodiments, the solubilizer is a polyglycol fatty monoester that may include at least one of each of ethylene glycol and propylene glycol units. In some embodiments, the glycol fatty monoester may include a copolymer of ethylene glycol and propylene glycol units. In some embodiments, the copolymer is a block copolymer. In other embodiments, the copolymer is a random copolymer.

In many embodiments, a polyglycol fatty monoester described herein may include any number of glycol units such that the solubilizer does not exceed an HLB greater than 10. For example, a polyglycol fatty monoester may include 1-4 glycol units. For example, a polyglycol fatty monoester may include 1, 2, 3, or 4 glycol units, or a value between a range of any of the preceding values, for example, between 1 and 3, between 1 and 2, or the like.

In some embodiments, the solubilizer is a glycerol fatty monoester.

In some embodiments, the solubilizer is a glycerol fatty monoester selected from glycerol monocaprylate, glycerol monocaprate, glycerol monolaurate, glycerol monomyristate, glycerol monopalmitate, glycerol monooleate, glycerol monostearate, and glycerol monoisostearate.

In some embodiments, the solubilizer is a glycerol fatty diester.

In some embodiments, the solubilizer is a glycerol fatty diester selected from glycerol dicaprylate, glycerol dicaprate, glycerol dilaurate, glycerol dimyristate, glycerol dipalmitate, glycerol dioleate, glycerol distearate, and glycerol diisostearate.

In some embodiments, the solubilizer is a glycerol fatty diester having fatty units independently selected from caprylate, caprate, laurate, myristate, palmitate, oleate, stearate, and isostearate.

In many embodiments, a glycerol fatty monoester described herein may include 1-4 glycerol units. For example, a glycerol fatty monoester may include 1, 2, 3, or 4 glycerol units, or a value between a range of any of the preceding values, for example, between 1 and 3, between 1 and 2, or the like.

In many embodiments, a glycerol fatty diester described herein may include 1-6 glycerol units. For example, a glycerol fatty diester may include 1, 2, 3, 4, 5, or 6 glycerol units, or a value between a range of any of the preceding values, for example, between 1 and 4, between 1 and 2, or the like.

In some embodiments, the solubilizer is a glycerol/glycol fatty mono- or di-ester that may include at least one glycerol unit and at least one glycol unit. For example, the glycol unit(s) may be selected from ethylene glycol and propylene glycol. In some embodiments, the glycerol/glycol fatty mono- or di-ester may include a copolymer of glycerol and one or more of ethylene glycol and propylene glycol units. In some embodiments, the copolymer is a block copolymer. In other embodiments, the copolymer is a random copolymer.

### Hydrophilic Vehicle

In some embodiments, any hydrophilic vehicle referred to herein may be characterized by having a hydrophilic-lipophilic balance greater than 10 as determined using the HLB Method.

In some embodiments, the hydrophilic vehicle may be selected from an alcohol. For example, the hydrophilic vehicle may be an C₁₋₄ alkanol or alkanol ether. Example alkanol and alkanol ethers include methanol, ethanol, isopropanol, glycerol, ethylene glycol, propylene glycol, methoxyisopropanol, or the like.

### Plasticizer

In some embodiments, the plasticizer may include a polyester polyol. For example, an amorphous polyester polyol may be derived from dimer diol or dimer acid.

### Pressure-Sensitive Adhesive (PSA)

In many embodiments, a PSA may include an acrylic polymer or copolymer. The acrylic polymer or copolymer may be prepared from at least one C₄₋₁₈ (cyclo)alkyl (meth)acrylate monomer, i.e., C₄₋₁₈ alkyl acrylate, C₄₋₁₈ cycloalkyl acrylate, C₄₋₁₈ alkyl methacrylate, or C₄₋₁₈ cycloalkyl methacrylate. In some embodiments, the acrylic polymer or copolymer may be prepared from at least one C₅₋₈ alkyl (meth)acrylate. In certain embodiments, the acrylic polymer or copolymer may be prepared from alkyl (meth)acrylate monomers having no greater than C₈ alkyl groups.

In some embodiments, a PSA acrylic polymer may be prepared from a C₅ alkyl (meth)acrylate monomer. In other embodiments, the acrylic polymer may be prepared from a C₈ alkyl (meth)acrylate monomer, e.g., isooctyl (meth)acrylate, 2-ethylhexyl (meth)acrylate, or the like.

In some embodiments a PSA acrylic copolymer may be prepared from at least one C₄₋₁₈ (cyclo)alkyl (meth)acrylate monomer, e.g., C₈ alkyl (meth)acrylate monomer, and at least one C₀₋₄ alkyl (meth)acrylate monomer. In some embodiments a PSA acrylic copolymer may be prepared from at least one C₄₋₁₈ (cyclo)alkyl (meth)acrylate monomer and at least one C₁₋₄ alkyl (meth)acrylate monomer. In some embodiments, the C₁₋₄ alkyl may be substituted with one or more hydroxyl group or one or more cyano group, e.g., C₁₋₄ hydroxyalkyl, C₁₋₄ cyanoalkyl, or the like. In some embodiments, the PSA acrylic copolymer may be prepared from at least one C₄₋₁₈ (cyclo)alkyl (meth)acrylate monomer and a C₄ alkyl (meth)acrylate monomer, e.g., butyl (methy)acrylate. In other embodiments, the PSA acrylic copolymer may be prepared from at least one C₄₋₁₈ (cyclo)alkyl (meth)acrylate monomer and a C₁₋₂ (meth)acrylate monomer, e.g., methyl (meth)acrylate, ethyl (meth)acrylate, or the like. In some embodiments, the PSA acrylic copolymer may be prepared from at least one C₄₋₁₈ (cyclo)alkyl (meth)acrylate monomer and a C₀ (methyl)acrylate monomer, i.e., acrylic acid or methacrylic acid.

In some embodiments, a PSA acrylic copolymer may be prepared from at least one C₄₋₁₈ (cyclo)alkyl (meth)acrylate monomer, e.g., C₈ alkyl (meth)acrylate monomer, and one or more monomer selected from (meth)acrylic acid, itaconic acid, maleic acid, fumaric acid, 2-hydroxyethyl (meth)acrylate, isobornyl (meth)acrylate, N-vinyl pyrrolidone, N-vinyl caprolactam, (meth)acylamide, N—C₁₋₄ alkyl (meth)acrylamide, N,N—C₁₋₄ dialkyl (meth)acrylamide, N—C₁₋₄ hydroxyalkyl (meth)acrylamide, N,N—C₁₋₄ dihydroxyalkyl (meth)acrylamide, N—C₁₋₄ alkyl, N—C₁₋₄ hydroxyalkyl (meth)acrylamide, (meth)acrylonitrile, and maleic anhydride, vinyl acetate, cyanoethyl (meth)acrylate, beta-carboxyethyl acrylate, vinyl neodecanoate, isoprenyl neodecanoate, vinyl neononanoate, isoprenyl neononanoate, vinyl neopentanoate, isoprenyl neopentanoate, vinyl 2-ethylhexanoate, isoprenyl 2-ethylhexanoate, vinyl propionate, isoprenyl propionate, vinylidene chloride, styrene, vinyl toluene, and C₁₋₆ alkyl vinyl ether.

In some embodiments, a PSA acrylic copolymer may be prepared from at least one C₄₋₁₈ (cyclo)alkyl (meth)acrylate monomer, e.g., C₈ alkyl (meth)acrylate monomer, and one or more monomer selected from (meth)acrylic acid, itaconic acid, maleic acid, fumaric acid, and 2-hydroxyethyl (meth)acrylate.

In some embodiments a PSA acrylic copolymer may be prepared from at least one C₄₋₁₈ (cyclo)alkyl (meth)acrylate monomer, e.g., C₈ alkyl (meth)acrylate monomer, and one or more monomer selected from N-vinyl pyrrolidone, N-vinyl caprolactam, (meth)acylamide, N—C₁₋₄ alkyl (meth)acrylamide, N,N—C₁₋₄ dialkyl (meth)acrylamide, N—C₁₋₄ hydroxyalkyl (meth)acrylamide, N,N—C₁₋₄ dihydroxyalkyl (meth)acrylamide, N—C₁₋₄ alkyl, N—C₁₋₄ hydroxyalkyl (meth)acrylamide, (meth)acrylonitrile, and maleic anhydride.

In some embodiments, a PSA acrylic copolymer may be prepared from a C₈ (cyclo)alkyl (methy)acrylate monomer, e.g., isooctyl acrylate, and N-vinyl pyrrolidone.

In some embodiments, a PSA acrylic copolymer may be prepared from at least one C₄₋₁₈ (cyclo)alkyl (meth)acrylate monomer having a homopolymer glass transition temperature that is less than the glass transition temperature of a co-monomer homopolymer. For example, co-monomers such as N-vinyl pyrrolidone, N-vinyl caprolactam, methacrylonitrile, and acrylic acid.

In some embodiments, a PSA acrylic copolymer may be prepared from at least one C₄₋₁₈ (cyclo)alkyl (meth)acrylate monomer having a homopolymer glass transition temperature that is greater than the glass transition temperature of a co-monomer homopolymer. For example, co-monomers such as ethoxyethoxyethyl acrylate (Tg=−71° C.) and methoxypolyethylene glycol 400 acrylate (Tg=−65° C.).

In other embodiments, a PSA comprises a block copolymer. In some embodiments, the block copolymer is a styrenic block copolymer, i.e., a block copolymer comprising at least one styrene hard segment, and at least one elastomeric soft segment. Exemplary styrenic block copolymers include dimmers such as styrene-butadiene (SB) and styrene-isoprene (SI). Additional exemplary styrenic block copolymers include styrene-isoprene-styrene (SIS), styrene-butadiene-styrene (SBS), styreneethylene/butadiene-styrene (SEBS), and styrene-ethylene/propylene-styrene block copolymers. In some embodiments, radial and star block copolymers may be used. Commercially available styrenic block copolymers include those available under the trade designation KRATON from Kraton Polymers LLC. including, e.g., KRATON D SBS and SIS block copolymers; and KRATON G SEBS and SEPS copolymers. Additional commercially available di- and tri-block styrenic block copolymers include those available under the trade designations SEPTON and HYBAR from Kuraray Co. Ltd., those available under the trade designation. In many embodiments, a PSA may include an acrylic polymer or copolymer. The acrylic polymer or copolymer may be prepared from at least one C₄₋₁₈ (cyclo)alkyl (meth)acrylate monomer, i.e., C₄₋₁₈ alkyl acrylate, C₄₋₁₈ cycloalkyl acrylate, C₄₋₁₈ alkyl methacrylate, or C₄₋₁₈ cycloalkyl methacrylate. In some embodiments, the acrylic polymer or copolymer may be prepared from at least one C₅₋₈ alkyl (meth)acrylate. In certain embodiments, the acrylic polymer or copolymer may be prepared from alkyl (meth)acrylate monomers having no greater than C₈ alkyl groups.

### Medical Articles

In many embodiments, a medical article is described. The medical article may include any octenidine-containing composition described herein and a drape backing.

In some embodiments, the drape backing may include a polyurethane film.

In some embodiments, the medical article may be an incise drape, a wound dressing, a tape, or the like.

In other embodiments, the medical article may be an incise drape.

### Methods

### Preparing Octenidine Solutions

In various embodiments, a method for preparing a homogenous solution is described. The method may include providing: an octenidine salt described herein and one or more solubilizer described herein. The method may include blending the octenidine salt and the one or more solubilizer to form a homogenous solution.

### Preparing Octenidine Drape Compositions

In various embodiments, a method for preparing an octenidine drape composition is described. The method may include providing: an octenidine salt described herein; a solubilizer described herein; a pressure-sensitive adhesive described herein; and a plasticizer described herein. The method may include blending the octenidine salt, the solubilizer, the pressure-sensitive adhesive, and the plasticizer to form an octenidine composition.

### Preparing Medical Articles

In various embodiments, a method for preparing a medical article (e.g., incise drape) is described. The method may include providing: an octenidine salt described herein; a solubilizer described herein; a pressure-sensitive adhesive described herein; and a plasticizer described herein. The method may include blending the octenidine salt, the solubilizer, the pressure-sensitive adhesive, and the plasticizer to form any of the drape compositions described herein. The method may include coating a release liner with the composition to form a wet-coated release liner and drying the wet-coated release liner under a set of conditions to form a dry-coated release liner. The method may include laminating the dry-coated release liner to a film to form the medical article.

In some embodiments, the release liner may have a wet adhesive thickness of about 2 to about 20 mils. The thickness may be in mils of about 2, 4, 6, 8, 10, 12, 14, 16, 18, or 20, or a value in a range between any of the preceding values, for example, between about 8 and about 12, between about 4 and about 16, or the like.

In some embodiments, the set of conditions may include drying the wet-coated release liner in an oven. The set of conditions may further include drying in an oven at a temperature of about 150° F. to about 200° F. The drying may be for a period between about 1 and about 10 minutes. For example, the drying period may be in min of about 1, 2, 3, 4, 5, 6, 7, 8, 9, or 10, or a value between a range between any of the preceding values, for example, between about 5 and 10, between about 3 and about 6, or the like. In some embodiments, the wet-coated release liner may be dried in an oven at 170° F. after about 10 min. In some embodiments, the drying may remove volatile components. The volatile components may include, for example, solvents from commercial pressure-sensitive adhesive solutions.

In some embodiments, the laminating may further include using nip rollers. Nip rollers may be used at a temperature of between about 20° C. to about 25° C.

### Medical Article Use

In various embodiments, a method for preparing a subject for disrupting a bodily surface. The method may include contacting a medical article (e.g., incise drape) described herein to the bodily surface of the subject. The medical article is in contact with the bodily surface during the disrupting of the bodily surface. The method may prevent the subject from contracting an infection at a disrupted bodily surface.

In some embodiments, the bodily surface is a skin surface.

In some embodiments, disrupting a bodily surface may include cutting, e.g., surgical procedures. In some embodiments, disrupting a bodily surface may include piercing, e.g., intravenous administrations or withdraws, installing or replacing drainage tubes, liposuction needle insertion, or the like.

### EXAMPLES

Objects and advantages of this invention are further illustrated by the following examples. The materials and amounts thereof recited in these examples, as well as other conditions and details, should not be construed as unduly limiting. These examples are merely for illustrative purposes and are not meant to limit the scope of the claims. All parts, percentages, ratios, etc. in the examples and the rest of the specification are by weight, unless noted otherwise. All reagents used were obtained from Sigma-Aldrich Chemical Company, St. Louis, Mo., unless otherwise noted.

### Materials

| | Source | |
|---|---|---|
| Materials | (Location) | Function |
| Random copolymer | 3M Company | Pressure-Sensitive |
| comprising 85 wt % | (St. Paul, MN) | Adhesive (PSA) |
| isooctyl acrylate (IOA) | | |
| and 15 wt % N-vinyl | | |
| pyrrolidone (NVP) | | |
| in methoxyisopropanol | | |
| Octenidine hydrochloride | Alfa Aesar (USA) | Bioactive Agent |
| Glycerol | Croda International | Solubilizer |
| monoisostearate (GMIS) | plc (UK) | |
| PRIPLAST ™ 3197 | Croda International | Plasticizer |
| (amorphous polyester | plc (UK) | |
| polyol derived from | | |
| dimer diol) | | |
| Propylene glycol | Abitec Corp. (USA) | Solubilizer |
| monoheptanoate (PGMH) | | |
| Thermoplastic | Lubrizol Corp. (USA) | Drape backing |
| polyurethane film 0.8 | | |
| mils, Estane 58309 | | |
| Chlorhexidine | Medichem SA (Spain) | Bioactive Agent |
| digluconate (CHG) 20% | | |
| w/v solution in water | | |

### Methods

### Octenidine Solutions

In all the examples, the solutions were prepared by dissolving finely divided octenidine hydrochloride (Dishman Pharma) in a solubilizer, at room temperature or elevated temperature depending on the functional signature, e.g., melting point, of the solubilizer.

### Octenidine Drape Compositions

Octenidine drape compositions were prepared by combining a PSA solution, an octenidine solution, and a plasticizer (or in a blend of plasticizers) through simple manual agitation.

Alternatively, the octenidine salt was directly dissolved in the pressure sensitive adhesive (PSA) solution prior to combining with the solubilizer and plasticizer. Solvent (hydrophilic vehicle) evaporation during coating and drying provided the octenidine salt solubilized in the plasticizer phase of the adhesive. Less than 1 wt % methoxyisopropanol remained with respect to the weight of the octendine salt after coating and drying.

### Coating

Octenidine drape compositions were coated as hand-spreads by applying a uniform layer on a suitable release surface, i.e., release liner, using a knife-edge coater to provide wet-coated release liners. The wet adhesive thickness ranged from 2-20 mils. The liners were dried in an oven for 1-10 minutes at temperatures between 65 and 95° C.

### Lamination

Dry-coated release liners were converted to medical articles by laminating the release liners with a suitable drape backing through nip rollers at room temperature.

### Direct Time Kill Testing of Antimicrobial Performance

Specimens of several Examples were subjected to antimicrobial performance testing. A 5-30 min. time kill study was performed as follows.

A suspension of methicillin-resistant Staphylococcus aureus (MRSA, ATCC #33592) was prepared at a concentration of 1×10⁸ colony forming units (CFU) per milliliter (mL) in phosphate-buffered water (pbw) using a 0.5 McFarland Equivalence Turbidity Standard. Using an Eppendorf pipette, 50 μL of this suspension (as 15-16 separate droplets) was transferred to a 2.5 cm diameter section of a surface coated with a dried octenidine drape composition. These inoculated specimens were then incubated at room temperature (23+/−2° C.) for 5-30 minutes. After incubation, the specimens were placed in 20 mL of neutralizing buffer and sonicated for one minute, followed by vortexing for two minutes. Portions of the resulting solution were serially diluted with pbw. The neat solution and dilutions were each plated to 3M PETRIFILM aerobic count plates (3M Company, St. Paul, Minn.) and incubated for at least 24 hours. The 3M PETRIFILM plates were then counted using a 3M PETRIFILM plate reader (model no. 6499, 3M Company).

### Example 1

Previous reports have shown that octenidine hydrochloride is soluble in both propylene glycol and ethylhexylglycerine, both of which are diol solvents. Hydrogen-bonding solvents with different functionalities were tested for their ability to solvate octenidine hydrochloride. Each solvent was combined with octenidine hydrochloride at 10% (w/w) with mixing at room temperature, i.e., 20-25° C. In some cases, sonication and/or heat were added to hasten dissolution. The results for solubilizing octenidine hydrochloride to form homogeneous solutions are reported in Table 1.

TABLE 1
Solubilization of octenidine hydrochloride in solvents.

| | Homogeneous |
|---|---|
| Solubilizer | solution |
| propylene glycol | Yes |
| glycerol monoisostearate | Yes |
| glycerol monocaprylate | Yes |
| methoxyisopropanol | Yes |
| propylene glycol monoheptanoate | Yes |
| propylene glycol monolaurate | Yes |
| lauramide diethanolamine | Yes |
| 2-octyldodecanol | No |
| propylene glycol dicaprylate/caprate | No |

Surprisingly, octenidine hydrochloride is soluble in glycol fatty monoesters, which are known to be ineffective solvents for chlorhexidine gluconate, i.e., glycol fatty monoesters do not form homogenous solutions with chlorhexidine gluconate. Interestingly, octenidine hydrochloride was insoluble in the glycol fatty diester and the monohydric alcohol, which suggests that octendine solubility is favorable in vehicles having at least two vicinal hydrogen-bonding groups with at least one group being a hydrogen-bond donor.

### Example 2

Antimicrobial octenidine drape compositions were prepared by manually agitation a pressure sensitive adhesive solution [25% PSA in solvent], a solution of octenidine hydrochloride (Octenid. HCl) in propylene glycol monoheptanoate (PGMH), GMIS, and PRIPLAST™ 3197. The compositions of these formulations on a solvent-free weight basis are provided in Table 2.

The octenidine drape compositions were applied on a siliconized release liner surface using a knife-edge coater in a uniform layer of 1 mil. Thickness and subsequently dried in an oven for 10 minutes at a temperature of 170° F. The dry-coated release liners were laminated to extruded film backings using nip rollers at room temperature to provide the incise drape.

TABLE 2
Antimicrobial octenidine drape compositions (% w/w).

| | | | | Octenid. | | PRIP. |
|---|---|---|---|---|---|---|
| Example | PSA | GMIS | CHG | HCl | PGMH | 3197 |
| Active 1 | 69.8 | 10 | 0 | 0.2 | 0 | 20 |
| Active 2 | 69 | 10 | 0 | 1.0 | 0 | 20 |
| Active 3 | 69.8 | 5 | 0 | 0.2 | 5 | 20 |
| Placebo | 70 | 5 | 0 | 0 | 5 | 20 |
| Comparative | 68 | 10 | 2 | 0 | 0 | 20 |
| example | | | | | | |

### Example 3

All the active and placebo drape samples were subject to a direct time-kill study. The test method was based on ASTM E2315-16 Standard Guide for Assessment of Antimicrobial Activity Using a Time-Kill Procedure.

A 6-log suspension of Staphylococcus aureus (ATCC 25923) was placed on the adhesive surfaces and incubated for 15 minutes at 35° C. After incubation the samples were dropped into a neutralizer and processed. Samples were diluted and plated for recovery of viable organisms. Neutralization was confirmed to be immediate and complete. The study rationale was twofold as follows. The results are provided in Table 3.
1. Compare octenidine drape samples to CHG drape controls.
2. Determine whether any positive benefit accrues from blending a superior solubilizing agent (propylene glycol monoheptanoate) with GMIS.

TABLE 3
Log reduction data for different actives and placebo
drape using direct time-kill on the adhesive surface

| | | Log | | | |
|---|---|---|---|---|---|
| | | reduction | Average | | |
| Drape | Log | (duplicate | log | CFU/test | CFU/test |
| sample | reduction | sample) | reduction | surviving | killed |
| Placebo | 0.16 | 0.00 | 0.08 | 831764 | 168236 |
| Active 1 | — | 0.42 | 0.42 | 380189 | 619811 |
| Active 2 | 4.81 | 3.24 | 4.03 | 93 | 999907 |
| Active 3 | 1.55 | 1.59 | 1.57 | 26915 | 973085 |
| Comparative | 0.79 | 0.86 | 0.83 | 147911 | 852089 |
| Example | | | | | |

The 1% octenidine drape (Active 2) is clearly more active than the Comparative Example (2% CHG drape), killing 1580 times as many CFU/test demonstrating both the availability of octenidine at the drape surface and the higher activity of octenidine at half the active concentration. In addition, the presence of a superior solubilizer (propylene glycol monoheptanoate) in the drape adhesive further boosts the availability and activity of octenidine in the drape (5× more active then CHG at one tenth the concentration).

### Equivalents

Those skilled in the art will recognize, or be able to ascertain, using no more than routine experimentation, numerous equivalents to the specific embodiments described specifically herein. Such equivalents are intended to be encompassed in the scope of the following claims.

1. A composition comprising:
a homogenous solution comprising:
a solubilizer comprising:
a pair of vicinal hydrogen-bonding groups, wherein at least one hydrogen-bonding group is a hydrogen-bond donor, and
a saturated or unsaturated C₇-C₂₂ hydrocarbon group;
an octenidine salt present in an amount greater than 0 wt % and up to about 20 wt % with respect to the weight of the solubilizer;
water present in an amount less than about 5 wt % with respect to the weight of the homogenous solution; and
a hydrophilic vehicle present in an amount in mols of less than about 2:1 with respect to the amount of octenidine salt,
wherein the octenidine salt is solubilized in the solubilizer to provide the homogenous solution at a temperature of about 20-25° C., or at a temperature equivalent to the melting point temperature of the solubilizer.

2. A composition comprising:
one or more solubilizer comprising:
a pair of vicinal hydrogen bonding groups, wherein one hydrogen bonding group is a hydrogen-bond donor, and
a saturated or unsaturated C₇-C₂₂ hydrocarbon group;
an octenidine salt present in an amount greater than 0 wt % and up to about 20 wt % with respect to the weight of the one or more solubilizer;
water present in an amount less than about 5 wt % with respect to the weight of the solubilizer and octenidine salt combined;
a hydrophilic vehicle present in an amount in mols of less than about 2:1 with respect to the amount of octenidine salt;
a pressure-sensitive adhesive; and
a plasticizer.

3. (canceled)

4. The composition of claim 2, wherein one hydrogen-bonding group is a hydrogen-bond acceptor; or wherein both hydrogen-bonding groups are hydrogen-bond donors.

5. (canceled)

6. The composition of claim 2, wherein the solubilizer is characterized by the formula:
R^{a}—W—R^{b},
wherein:
R^{a} is an C₂₋₆ alkyl, C₆₋₁₂ aralkyl, C₆₋₁₀ alkaryl, or C₆₋₁₀ aryl, wherein the C₂₋₆ alkyl, C₆₋₁₂ aralkyl, C₆₋₁₀ alkaryl, or C₆₋₁₀ aryl is substituted with one or more of —OH and —N(R¹)(R²), and is optionally substituted with —W—R^{b};
each W is independently selected from —O—, —N(R³)—, —O—C(O)—, —C(O)—O—, —O—C(O)—O—, —O—C(O)—N(R³)—, —N(R³)—C(O)—O—, —N(R³)—C(O)—, —C(O)—N(R³)—, or —N(R³)—C(O)—N(R³)—;
R^{b} is a C₇-C₂₂ hydrocarbon group selected from C₇₋₂₂ alkyl and C₇₋₂₂ alkenyl, wherein the C₇₋₂₂ alkyl and C₇₋₂₂ alkenyl are optionally substituted with one or more hydroxyl;
R¹, R², and R³ are independently selected from —H, C₁₋₆ alkyl, 2-hydroxy C₂-C₆ alkyl, 2-amino C₂-C₆ alkyl.

7. The composition of claim 2, wherein the solubilizer is selected from a glycol fatty monoester, an ethanolamine fatty monoester, an ethanolamine fatty monoamide, an ethylenediamine fatty monoamide, a glycerol fatty monoester, a glycerol fatty diester, an aminopropanediol fatty monoester, an aminopropanediol fatty monoamide, a diaminopropanol fatty monoamide, and a diaminopropanol fatty monoester.

8. The composition of claim 7, wherein the solubilizer is a glycol fatty monoester selected from propylene glycol monocaprylate, propylene glycol monocaprate, propylene glycol monoheptanoate, propylene glycol monolaurate, propylene glycol monomyristate, propylene glycol monopalmitate, propylene glycol monooleate, propylene glycol monostearate, and propylene glycol monoisostearate
or
wherein the solubilizer is a glycol fatty monoester selected from ethylene glycol monocaprylate, ethylene glycol monocaprate, ethylene glycol monoheptanoate, ethylene glycol monolaurate, ethylene glycol monomyristate, ethylene glycol monopalmitate, ethylene glycol monooleate, ethylene glycol monostearate, and ethylene glycol monoisostearate; or
wherein the solubilizer is a glycerol fatty monoester selected from glycerol monocaprylate, glycerol monocaprate, glycerol monolaurate, glycerol monomyristate, glycerol monopalmitate, glycerol monooleate, glycerol monostearate, and glycerol monoisostearate.

9-10. (canceled)

11. The composition of claim 2, wherein the octenidine salt is octenidine hydrochloride.

12. The composition of claim 2, wherein the hydrophilic vehicle is selected from glycerol, ethylene glycol, propylene glycol, C₁-C₄ alkanol, methoxyisopropanol, and a combination thereof.

13. The composition of claim 2, wherein:
the octenidine salt is present in an amount up to about 2 wt %;
the solubilizer is a glycol fatty monoester, the glycol fatty monoester is present in an amount of about 5 wt % to about 10 wt %;
the pressure-sensitive adhesive is present in an amount of about 65 wt % to about 75 wt %; and
the plasticizer is present in an amount of about 15 wt % to about 25 wt %,
wherein each wt % is with respect to the weight of the composition.

14. The composition of claim 2, the pressure-sensitive adhesive comprising an aliphatic (meth)acrylate polymer and an N-vinylpyrrolidone polymer.

15. The composition of claim 2, wherein the plasticizer selected from a polyester polyol, polyether polyol, polyamide polyol, and copolymers thereof.

16. The composition of claim 2, further comprising a second solubilizer present in an amount of about 5 wt % to about 10 wt %, wherein wt % is with respect to the weight of the composition, and wherein the second solubilizer is a glycerol fatty monoester.

17. A medical article comprising:
an antimicrobial adhesive composition comprising a composition of claim 2; and
a drape backing.

18. The medical article of claim 17, the drape backing comprising a polyurethane film.

19. The medical article of claim 17, being an incise drape.

20. A method of preparing a medical article of claim 17, the method comprising:
providing:
an octenidine salt,
a solubilizer comprising:
a pair of vicinal hydrogen-bonding groups, wherein at least one hydrogen-bonding group is a hydrogen-bond donor, and
a saturated or unsaturated C₇-C₂₂ hydrocarbon group;
a pressure-sensitive adhesive, and
a plasticizer;
blending the octenidine salt, the solubilizer, the pressure-sensitive adhesive, and the plasticizer to form a composition;
coating a release liner with the composition to form wet-coated release liner;
drying the wet-coated release liner under a set of conditions to form a dry-coated release liner; and
laminating the dry-coated release liner to a drape backing to form the medical article.

21. The method of claim 20, the set of conditions comprising drying the wet-coated release liner in an oven for about 10 min. at a temperature of about 170° C.

22. The method of claim 20, the laminating further comprising using nip rollers at a temperature of between about 20° C. to about 25° C.

23. The composition of claim 1, wherein the solubilizer is characterized by the formula:
R^{a}—W—R^{b},
wherein:
R^{a} is an C₂₋₆ alkyl, C₆₋₁₂ aralkyl, C₆₋₁₀ alkaryl, or C₆₋₁₀ aryl, wherein the C₂₋₆ alkyl, C₆₋₁₂ aralkyl, C₆₋₁₀ alkaryl, or C₆₋₁₀ aryl is substituted with one or more of —OH and —N(R¹)(R²), and is optionally substituted with —W—R^{b};
each W is independently selected from —O—, —N(R³)—, —O—C(O)—, —C(O)—O—, —O—C(O)—O—, —O—C(O)—N(R³)—, —N(R³)—C(O)—O—, —N(R³)—C(O)—, —C(O)—N(R³)—, or —N(R³)—C(O)—N(R³)—;
R^{b} is a C₇-C₂₂ hydrocarbon group selected from C₇₋₂₂ alkyl and C₇₋₂₂ alkenyl, wherein the C₇₋₂₂ alkyl and C₇₋₂₂ alkenyl are optionally substituted with one or more hydroxyl;
R¹, R², and R³ are independently selected from —H, C₁₋₆ alkyl, 2-hydroxy C₂-C₆ alkyl, 2-amino C₂-C₆ alkyl.

24. The composition of claim 1, wherein the hydrophilic vehicle is selected from glycerol, ethylene glycol, propylene glycol, C₁-C₄ alkanol, methoxyisopropanol, and a combination thereof.
